(19) United States
(12) Patent Application Publication
(10) Pub. No.: US 2023/0414542 A1
(43) Pub. Date: Dec. 28, 2023

(54) ORALLY DISPERSIBLE COMPOUND CONTAINING AN ESTER OR SALT OF N-BUTYRIC ACID AND PROCESS FOR PRODUCTION

(71) Applicant: SILA SRL, Noale (VE) (IT)

(72) Inventor: Maurizio LORENZON, Noale (VE) (IT)

(73) Assignee: SILA SRL, Noale (VE) (IT)

(21) Appl. No.: 18/036,482

(22) PCT Filed: Nov. 12, 2021
(86) PCT No.: PCT/IB2021/060505

§ 371 Date: May 11, 2023

(30) Foreign Application Priority Data
Nov. 13, 2020 (IT) 102020000027305

(51) Int. Cl. A61K 31/19; A61K 9/00; A61K 9/28; A61K 9/20

(52) U.S. Cl. CPC A61K 31/19; A61K 9/0056; A61K 9/2813; A61K 9/282; A61K 9/286; A61K 9/2826; A61K 9/2893; A61K 9/2886; A61K 9/205; A61K 9/2018

(57) ABSTRACT

An orally dispersible compound, which contains at least one ester or a salt of n-butyric acid, includes a granular material including at least one ester or salt of n-butyric acid and having a first and a second coating, and at least one excipient which is capable of making the granular material orally dispersible. The first coating includes from 20.0% to 99.0% by weight with respect to the first coating of saturated fatty acids with a C₁₄-C₂₂ long chain, and the second coating includes at least one thickening agent, the first coating being between the granular material and the second coating.

### TECHNICAL FIELD

The present invention relates to an orally dispersible compound which contains an ester or a salt of n-butyric acid and which is intended mainly for human consumption, having the features set out in the preamble of the main claim and a corresponding process for producing it.

### TECHNOLOGICAL BACKGROUND

It is known that some compounds of n-butyric acid, in particular the esters and salts thereof, have interesting biological effects on the digestive system, stimulating the growth of the intestinal villi and modifying the development of the enteric microorganisms.

These compounds, which are generally defined as butyrates, depending on the surrounding environment, may be in dissociated form or in non-dissociated form, the latter form being the most effective.

As known, however, the compounds of n-butyric acid are particularly sensitive to acidic environments where they can readily hydrolyse and reform the original n-butyric acid, characterized by the particularly unpleasant odour of rancid butter.

For this reason, if the butyrate is administered per se to an animal or to a human being, the n-butyric acid would immediately be formed at a gastric level, making it no longer available for the absorption thereof at an intestinal level.

In order to limit this disadvantage, there is proposed in EP2352386 A1 a method of microencapsulation of the compounds of n-butyric acid which provides for coating these compounds with a suitable lipid matrix.

For the purposes of human administration, the product obtained in this manner is usually enclosed in an operculum which allows the product itself to be readily swallowed and which protects it further from premature gastric degradation in n-butyric acid.

Disadvantageously, the opercula may in some cases be difficult to swallow.

This may occur, for example, in the case of the absence of water or during travel, or generally for some sections of the population such as the elderly and young sections, or in the case of some pathologies, as may be the case with dysphagia and odynophagia. Therefore, there remains in the technical field being referred to a requirement to provide a product based on a compound of n-butyric acid which can be readily administered and which, at the same time, is gastro-resistant and a process for producing the compound itself.

### STATEMENT OF INVENTION

The problem addressed by the present invention is to provide a product based on a compound of n-butyric acid which is functionally configured to overcome the limitations set out above with reference to the cited prior art.

In the context of this problem, an object of the invention is to provide a product based on a compound of n-butyric acid which allows a release of the active ingredient in the intestine in a slow and controlled manner, but which is in a readily administrable form.

Another object of the invention is to provide a process for producing this product.

This problem is solved and these objects are achieved by the present invention by means of an orally dispersible product which comprises a compound of n-butyric acid and a process for obtaining it according to the appended claims.

In a first aspect thereof, therefore, the present invention is directed towards an orally dispersible compound which contains at least one ester or a salt of n-butyric acid, comprising:
a granular material comprising the at least one ester or salt of n-butyric acid and having a first and a second coating, and
at least one excipient which is arranged on the surface of the granular material and which is capable of dissolving in the oral cavity within a maximum time of 3 minutes, preferably 2 minutes, and making the granular material orally dispersible, the first coating comprising from 20.0% to 99.0% by weight with respect to the first coating of saturated fatty acids with a C₁₄-C₂₂ long chain, and the second coating comprising at least one thickening agent, the first coating being between the granular material and the second coating.

This allows the provision of a compound which comprises at least one ester or one salt of n-butyric acid which can readily be consumed and which can therefore be dissolved in the mouth without any need to have to swallow any operculum, and without any need for water. Advantageously, this makes it easier its uptake and provides greater freedom to the patient. Furthermore, some types of subjects also benefit therefrom, such as the elderly or children, or those having difficulty in swallowing. At the same time, as a result of the second coating, once the compound is dissolved in the mouth, the compound based on the at least one ester or salt of n-butyric acid does not suffer premature gastric degradation in n-butyric acid, something which could occur in the absence of that coating, making it unavailable for the absorption thereof at an intestinal level.

In a second aspect thereof, the present invention relates to a process for preparing an orally dispersible compound containing at least one ester or salt of n-butyric acid, comprising the steps of:
providing a granular material comprising the at least one ester or salt of n-butyric acid having a first coating comprising from 20.0% to 99.0% by weight with respect to the first coating of saturated fatty acids with a C₁₄-C₂₂ long chain;
recoating the granular material having the first coating with a second coating comprising at least one thickening agent;
granulating the granular material comprising the first coating and the second coating with at least one excipient which is capable of dissolving in the oral cavity within a maximum time of 3 minutes, preferably 2 minutes, and of making the granular material orally dispersible.

When the granular material comprising the first coating and the second coating is granulated with this excipient, this latter is arranged on the surface of the granular material.

Advantageously, this process allows the production of a compound of at least one ester or a salt of n-butyric acid which is readily consumable but which, at the same time, does not suffer premature degradation in n-butyric acid once the gastric tract has been reached.

In the present description, as in the claims appended thereto, some terms and expressions are considered to have, unless explicitly indicated otherwise, the meaning expressed in the following definitions.

The term “excipient which is capable of making the granular material orally dispersible” is intended to be understood to be any excipient which is capable of being dissolved in the oral cavity, such as, for example, polysaccharides and polyols, and sweeteners in general, within a maximum time of 3 minutes, preferably 2 minutes.

In particular, the term “orally dispersible compound” is intended to be understood to be a compound which becomes separated or disperses rapidly in the oral cavity without using water, but instead only with the saliva which is normally present in the oral cavity.

In the context of the present invention, it is the presence of this excipient which is arranged on the surface of the granular material making the compound orally dispersible.

This excipient, in contact with saliva present in the oral cavity, dissolves, at the same time stimulating the production of additional saliva. This saliva then allows the granular material to readily pass into the stomach, where the second coating protects the compound based on at least one ester or salt of n-butyric acid from premature gastric degradation in n-butyric acid, therefore preventing it from no longer being available for the absorption thereof at an intestinal level.

The person skilled in the art is capable of selecting an excipient which can make the granular material orally dispersible in the context of the normal formulation activity. The FDA of the United States defines as an “orally disintegrating tablet” or “orally dissolving tablet” ODT a type of solid dosage which contains medicinal substances and which disintegrates rapidly, usually within a few seconds, when it is placed on the tongue. The European Pharmacopoeia has also adopted the term “orally dispersible pill” as a pill which has to be placed in the mouth where it rapidly disperses.

With reference to saturated fatty acids with a C₁₄-C₂₂ long chain, the term “saturated” is intended to be understood to indicate fatty acids having a level of saturation of at least 99.0%. Furthermore, it is preferable for the fatty acids present to be substantially present in the form of glycerides and not free acids, in particular it is preferable for the percentage of free acids within the lipid component of the matrix to be less than 10.0% by weight and more preferable for it to be less than 1.0% by weight.

Preferably, the glycerides are in the form of triglycerides.

Preferably, the lipid component of the matrix according to the invention further has a content of saturated fatty acid C₁₈ between 20.0% and 50.0% by weight and a content of saturated fatty acid C₁₆ between 40.0% and 70.0% by weight with respect to the total of the saturated fatty acids which constitute the glycerides.

Preferably, the lipid component of the matrix is based on hydrogenated palm oil.

In some embodiments, the at least one ester or salt of n-butyric acid is a salt of n-butyric acid, preferably it is the sodium salt of n-butyric acid.

Advantageously, the sodium salt of n-butyric acid is the compound of n-butyric acid which has the greatest biological effects on the digestive system.

Preferably, the first coating comprises from 60.0% to 97.0%, more preferably from 75.0% to 95.0% by weight with respect to the first coating of saturated fatty acids with a C₁₄-C₂₂ long chain.

In some embodiments, the first coating comprises from 0.5% to 20.0% by weight of a mineral filler, preferably from 1.0% to 15.0% by weight with respect to the first coating.

The term “mineral filler” is intended to be understood to be any inorganic solid substance which is finely subdivided with a characteristic crystalline form.

In some embodiments, this mineral filler has a fraction of calcium sulphate dihydrate between 0.2% and 5.0% by weight, preferably between 0.5% and 2.5% by weight with respect to the first coating.

The calcium sulphate dihydrate, CaSO₄·2(H₂O), further improves the resistance of the granular compound to the extremely acidic environment present at a gastric level, protecting the product during the digestive phase.

In some embodiments, the first coating comprises from 0.01% to 5.0% by weight of one or more essential oils, preferably from 0.1% to 2.0% by weight with respect to this first coating.

Essential oils have the purpose of flavourings, antioxidants and antibacterial agents, at the same time enhancing the individual antibacterial activity of the butyrate.

In general, the term “thickening agent” is intended to be understood to be any agent capable of increasing the consistency and/or the density and/or the stability of a predetermined product.

The at least one thickening agent of the second coating is preferably selected from the group consisting of rubbers, alginates, starches and modified starches, flours, algae and products derived from algae, polyols and polysaccharides.

In some embodiments, the at least one thickening agent of the second coating comprises guar gum, corn starch and sorbitol.

Advantageously, the presence of the guar gum, in addition to improving the stability of the compound, further improves the efficacy of the compound based on the at least one ester or salt of n-butyric acid, being a fibre which activates the lower intestine, where it is fermented with fatty acids with a short chain, in particular stimulating the endogenous production of n-butyric acid.

Preferably, the second coating comprises from 0.5% to 10.0% by weight of guar gum, more preferably from 1.0% to 5.0% by weight, with respect to the second coating.

Preferably, the second coating comprises from 30.0% to 90.0% by weight of corn starch, more preferably from 50.0% to 70.0% by weight with respect to the second coating.

Advantageously, this compound acts as a thickening, gelling and stabilizing agent, increasing the consistency of the compound.

The second coating preferably comprises from 10.0% to 60.0% by weight of sorbitol, more preferably from 30.0% to 40.0% by weight with respect to the second coating.

Advantageously, the sorbitol acts as a sweetening, binding and stabilizing agent, increasing the consistency of the compound; furthermore, it increases the retention of humidity inside the compound and improves the conservation thereof.

Furthermore, the sorbitol increases the thickening capacity of the corn starch.

In some embodiments, the second coating comprises sodium alginate, preferably from 0.5% to 10.0% by weight, more preferably from 1.0% to 5.0% by weight with respect to the second coating.

Advantageously, sodium alginate is a thickening and gelling agent which facilitates the formation of the second coating. It further acts in synergy with the guar gum because it enhances the thickening capacity thereof.

Preferably, the excipient which is capable of making the granular material orally dispersible and which has the first and second coatings comprises isomalt and guar gum.

In some embodiments, the at least one excipient which is capable of making the granular material orally dispersible and which has the first and second coatings comprises:
from 30.0% to 90.0% by weight of isomalt with respect to the orally dispersible compound, and
from 0.1% to 10.0% by weight of guar gum with respect to the orally dispersible compound.

Preferably, the at least one excipient which is capable of making the granular material orally dispersible and which has the first and second coatings comprises from 50.0% to 80.0% by weight, more preferably from 55.0% to 75.0% by weight, of isomalt with respect to the orally dispersible compound.

Isomalt, being a sweetening agent, increases the compliance of the orally dispersible compound; at the same time, with respect to saccharose, it has a lower calorific value (2 kcal/g) and is non-cariogenic. It further has a low glycaemic index, greater thermal stability and lower hygroscopicity.

Preferably, the at least one excipient which is capable of making the granular material orally dispersible and which has the first and second coatings comprises from 0.5% to 7.0% by weight, more preferably from 1.0% to 5.0% by weight, of guar gum with respect to the orally dispersible compound.

According to an embodiment, the orally dispersible compound may comprise variable quantities between 0.1% and 5.0% by weight with respect to this orally dispersible compound, preferably between 0.5% and 3.0% by weight, one or more flavouring agents such as, by way of non-limiting example, orange flavouring.

In an embodiment, the orally dispersible compound may comprise from 0.01% to 4.0% by weight of stevia, preferably from 0.1% to 2.0% by weight, with respect to this orally dispersible compound.

Stevia is used as a result of its high sweetening power and the absence of any calorific contribution.

In some embodiments, the process for preparing this orally dispersible compound comprises the steps of:
providing a granular material comprising the at least one ester or salt of n-butyric acid having a first coating comprising from 20.0% to 99.0% by weight with respect to the first coating of saturated fatty acids with a C₁₄-C₂₂ long chain;
recoating the granular material having the first coating with a second coating comprising at least one thickening agent, the at least one thickening agent comprising guar gum, corn starch and sorbitol;
granulating the granular material having the first coating and the second coating with at least one excipient which is capable of dissolving in the oral cavity within a maximum time of 3 minutes and making the granular material orally dispersible, the at least one excipient comprising isomalt and guar gum.

In some embodiments, the process for recoating the granular material having the first coating with a second coating comprises the step of forming the second coating by adsorption of a first aqueous solution comprising guar gum and a second aqueous solution comprising corn starch and sorbitol on the granular material having the first coating.

Preferably, the first aqueous solution comprising guar gum also comprises sodium alginate.

Preferably, the step of recoating the granular material having the first coating with the second coating is carried out in a fluid-bed granulator.

Preferably, the granulation step is carried out in a fluid-bed granulator.

By way of non-limiting example, the granular material comprising the at least one ester or salt of n-butyric acid can be obtained according to the description in EP2352386 A1. The first coating is applied to this granular material which is powdered and sliding.

By way of non-limiting example, the first coating can also be obtained according to the description in EP2352386 A1.

The granular material comprising the first coating may possibly be subjected to suitable sieving so as to eliminate the granular material having this first coating but dimensions greater than a specific desired value before the second coating is carried out.

The second coating on the granular material having this first coating can be carried out by means of a fluid-bed granulation technique. Merely by way of non-limiting example, for example, a first and a second aqueous solution are prepared. The first aqueous solution is prepared, preferably in the cold state, by mixing water and from 0.3% to 1.0% by weight of guar gum. Subsequently, this mixture is brought to a temperature between 70° C. and 90° C., preferably to 80° C., and there is added from 0.3% to 1.0% by weight of sodium alginate. The second aqueous solution is prepared, preferably in the cold state, by mixing water, from 5.0% to 10.0% by weight of sorbitol and from 10.0% to 15.0% by weight of corn starch. Subsequently, this mixture is brought to a temperature between 60° C. and 80° C., preferably to 70° C.

In a fluid-bed granulator, there are successively atomized on the granular material having the first coating the first aqueous solution having a temperature between 70° C. and 90° C., preferably at 80° C., and the second aqueous solution having a temperature between 60° C. and 80° C., preferably at 70° C.

The introduction temperature of the granular material having the first coating is between 20° C. and 45° C. and it is preferably 35° C. The air being introduced into the granulator has a temperature between 25° C. and 45° C. and it is preferably 35° C. The air further has a relative humidity between 0.5% and 2.0%, and it is preferably 1.0%.

This process typically lasts from 1 to 4 hours, preferably 2 hours, at the end of which there is obtained a core which is constituted by the granular material comprising the at least one ester or salt of n-butyric acid, a first coating on the core and a second coating on the first coating comprising sorbitol, corn starch, sodium alginate and guar gum.

Subsequently, the temperature of the air being introduced into the granulator is brought to a value in the range between 40° C. and 65° C., and this value is preferably 55° C., until obtaining a residual relative humidity in the granular material having the first coating and the second coating of 1.0%, or in any case continues for a time between 10 and 40 minutes.

Preferably, the granulation step is carried out in a fluid-bed granulator. In particular, by way of non-limiting example, the granular material having the first coating and the second coating can be mixed together with isomalt, stevia and guar gum.

In some cases, the granular material having this first coating and second coating can be discharged from the fluid-bed granulator and sieved so as to eliminate the material with dimensions greater than a specific desired value before being introduced therein again or in another fluid-bed granulator in order to be mixed together with isomalt, stevia and guar gum.

The temperature of the air being introduced into the granulator is in the range between 60° C. and 70° C., and it is preferably 65° C. The mixing is continued until obtaining a residual relative humidity of 1.0%, and in any case for no less than 10 minutes. Once this humidity value has been reached, there are optionally added the flavourings and the product is mixed for a further 5 minutes.

The entire granulation process typically lasts for from 20 to 50 minutes, preferably 30 minutes, at the end of which there is obtained an orally dispersible granular material having a core which comprises the at least one ester or salt of n-butyric acid, a first coating on the core and a second coating on the first coating.

The orally dispersible granular material is then discharged from the fluid-bed granulator and can be sieved on a suitable mesh so as to eliminate the granules having dimensions greater than a specific desired value.

### EMBODIMENTS

### First Coating

In a first example, the first coating was prepared in accordance with EP2352386 A1.

The composition of the granular material having a first coating in accordance with this example is set out in Table 1.

TABLE 1
Composition of the granular material having a first coating

| | Component | % by weight |
|---|---|---|
| Granular material | Sodium butyrate | 50.8 |
| First coating | Saturated fatty acids C₁₄-C₂₂ | 45.5 |
| | Calcium carbonate | 2.5 |
| | Calcium stearate | 0.5 |
| | Calcium sulphate dihydrate | 0.5 |
| | Flavouring | 0.2 |

### Second Coating

### Preparation of the First Aqueous Solution A

There were mixed at ambient temperature water and guar gum at concentrations of 0.6% by weight with respect to the total of the solution. Subsequently, this mixture was brought to 80° C. and 0.6% by weight of sodium alginate with respect to the total of the solution was added.

### Preparation of the Second Aqueous Solution B

Water, 7.5% by weight of sorbitol and 12.5% by weight of corn starch with respect to the total of the solution were mixed at ambient temperature; the solution was then brought to 70° C.

### Application of the Second Coating

In a fluid-bed granulator, there were successively atomized on the granular material comprising sodium butyrate and the first coating and having an introduction temperature into the fluid bed of 35° C. the solutions A and B at 80° C. and 70° C., respectively. The air flow being introduced into the granulator had a temperature of 35° C. and a relative humidity of 1.0%.

This process lasted 2 hours at the end of which there is obtained a granular material comprising sodium butyrate, a first coating on this granular material and a second coating comprising sorbitol, corn starch, sodium alginate and guar gum on this first coating.

Subsequently, the temperature of the air being introduced into the granulator was brought to 55° C. until obtaining a residual relative humidity in the granular material of 1.0%, established on suitable samples of the material itself from the granulator. The composition of the granular material having this first coating and second coating obtained in this manner is set out in Table 2.

TABLE 2
Composition of the granular material having a first coating
and a second coating

| | Component | % by weight |
|---|---|---|
| | Granular material with first coating | 92.6 |
| Second | Sodium alginate | 0.2 |
| coating | Guar gum | 0.2 |
| | Sorbitol | 2.6 |
| | Corn starch | 4.4 |

### Granulation

The granular material having a first coating and a second coating being discharged from the fluid-bed granulator was sieved over a suitable mesh so as to eliminate the granules having a diameter greater than 2 mm.

The granular material having this double coating and a diameter below 2 mm was again conveyed to the fluid-bed granulator and mixed together with isomalt, stevia and guar gum. The temperature of the air being introduced was brought to 65° C. and the mixing was continued until having a residual relative humidity in the granular material of 1.0%. Subsequently, the flavourings were added and mixing was continued for another 5 minutes.

The orally dispersible compound obtained in this manner was then discharged from the fluid-bed granulator and sieved over a suitable mesh so as to eliminate the granules having a diameter greater than 2 mm.

The composition of the orally dispersible compound obtained in this manner is set out in Table 3.

TABLE 3
Composition of the orally dispersible compound comprising a granular
material having a first coating and a second coating

| Component | % by weight |
|---|---|
| Granular material having a first coating and a second | 29.0 |
| coating | |
| Isomalt | 67.7 |
| Stevia | 0.2 |
| Guar gum | 2.0 |
| Flavourings | 1.1 |

### Oral Dispersibility Tests

In order to evaluate the oral dispersibility of the composition to which the present invention relates, there was proposed a test in vivo according to the following description.

A quantity of orally dispersible compound of 1.5 grammes, which is indicated A in the table below and which has a composition according to what is set out in Table 3, was placed on the tongue of three different subjects who were asked to keep it still to the greatest possible extent. At predetermined intervals of time of 15 seconds, a visual examination of the compound itself was carried out, by asking the subjects to open their mouths.

The same type of test was carried out with the granular material which has a first coating and a second coating, which is indicated B and which has a composition according to what is set out in Table 2.

In this case, there were used both 1.5 g of the granular material B and 435 mg, that is to say, 29% of 1.5 g, equal to the quantity of the granular material B present in A used in the above test. The results obtained in the two cases were identical; therefore, the Table below sets out a single result for the granular material B.

Subject 1 is a male aged 31, subject 2 is a female aged 27 and subject 3 is a female aged 28.

Table 4 sets out the results of the observations, where A is intended to denote the orally dispersible compound and B is intended to denote the granular material having the first coating and the second coating.

TABLE 4
Results of the tests in vivo

| Subject | | 45 sec | 2 min | 3 min |
|---|---|---|---|---|
| 1 | A | Became separated | No longer any | |
| | | | trace of A | |
| | B | | | The granules of B |
| | | | | are still present |
| 2 | A | Became partially | | No longer any |
| | | separated | | trace of A |
| | B | | | The granules of B |
| | | | | are still present |
| 3 | A | Became partially | | No longer any |
| | | separated | | trace of A |
| | B | | | The granules of B |
| | | | | are still present |

Though the subjects of the test differ in terms of sex and age, and similarly in terms of extent of the surface of the tongue, composition and flow of saliva, this test makes it clear that it was possible for all three subjects to take up the orally dispersible compound very easily and without using water within 3 minutes, while none of the three subjects was able to take up the granular material having the first coating and second coating without using water.

1. An orally dispersible compound, which contains at least one ester or a salt of n-butyric acid, comprising:
a granular material comprising the at least one ester or salt of n-butyric acid and having a first and a second coating, and
at least one excipient which is arranged on the surface of the granular material and which is dissolvable in the oral cavity within a maximum time of 3 minutes and which is capable of making the granular material orally dispersible,
the first coating comprising from 20.0% to 99.0% by weight with respect to the first coating of saturated fatty acids with a C₁₄-C₂₂ long chain, and the second coating comprising at least one thickening agent, the first coating being between the granular material and the second coating.

2. The orally dispersible compound according to claim 1, wherein the at least one thickening agent of the second coating is selected from the group consisting of:
rubbers, alginates, starches, modified starches, flours, algae, products derived from algae, polyols and polysaccharides.

3. The orally dispersible compound according to claim 2, wherein the at least one thickening agent comprises guar gum, corn starch and sorbitol.

4. The orally dispersible compound according to claim 3, wherein the at least one thickening agent comprises from 0.5% to 10.0% by weight of guar gum, from 30.0% to 90.0% by weight of corn starch and from 10.0% to 60.0% by weight of sorbitol.

5. The orally dispersible compound according to claim 1, wherein the at least one excipient which is capable of making the granular material orally dispersible comprises isomalt and guar gum.

6. The orally dispersible compound according to claim 2, wherein the at least one thickening agent of the second coating comprises sodium alginate.

7. The orally dispersible compound according to claim 1, wherein the first coating comprises from 0.5% to 20.0% by weight of a mineral filler.

8. The orally dispersible compound according to claim 7, wherein the mineral filler comprises between 0.2% and 5.0% by weight of calcium sulphate dihydrate with respect to the first coating.

9. The orally dispersible compound according to claim 1, wherein the at least one excipient which is capable of making the granular material orally dispersible comprises:
from 30.0% to 90.0% by weight of isomalt, with respect to the orally dispersible compound, and
from 0.1% to 10.0% by weight, of guar gum with respect to the orally dispersible compound.

10. A process for preparing an orally dispersible compound according to claim 1 comprising:
providing a granular material comprising the at least one ester or salt of n-butyric acid having a first coating comprising from 20.0% to 99.0% by weight with respect to the first coating of saturated fatty acids with a C₁₄-C₂₂ long chain;
recoating the granular material having the first coating with a second coating comprising at least one thickening agent;
granulating the granular material comprising the first coating and the second coating with at least one excipient which is dissolvable in the oral cavity within a maximum time of 3 minutes and which is capable of making the granular material orally dispersible.

11. The process for preparing an orally dispersible compound according to claim 10, further comprising:
providing a granular material comprising the at least one ester or salt of n-butyric acid having a first coating comprising from 20.0% to 99.0% by weight with respect to the first coating of saturated fatty acids with a long C₁₄-C₂₂ chain;
recoating the granular material having the first coating with a second coating comprising guar gum, corn starch and sorbitol;
granulating the granular material having the first coating and the second coating with isomalt and guar gum.

12. The process for preparing an orally dispersible compound according to claim 10, wherein the step of recoating the granular material having the first coating with the second coating is carried out in a fluid-bed granulator.

13. The process for preparing an orally dispersible compound according to claim 10, wherein the granulation is carried out in a fluid-bed granulator.
